(19) United States
(12) Patent Application Publication
(10) Pub. No.: US 2007/0207520 A1
(43) Pub. Date: Sep. 6, 2007

(54) Method for cell-free synthesizing protein using wheat embryo extract under low temperature condition

(76) Inventors: Ryo Morishita, Yokohama-shi (JP); Aya Kitamoto, Yokohama-shi (JP)

Correspondence Address: KILYK & BOWERSOX, P.L.L.C., 400 HOLIDAY COURT, SUITE 102, WARRENTON, VA, 20186, US

(21) Appl. No.: 11/712,039

(22) Filed: Feb. 28, 2007

(30) Foreign Application Priority Data
Mar. 2, 2006 (JP) JP2006-056835

(51) Int. Cl. C12P 21/06

(52) U.S. Cl. 435/68.1

(57) ABSTRACT

The subject of the present invention is to provide a method for cell-free protein synthesis which needs no new apparatus and reagent to allow production of a protein with a high quality by a large amount. To solve the matter described above, translation reaction is conducted at a temperature of 0° C.-20° C., which is lower than approximately 26° C., that is, a temperature suitable for a conventionally known method for cell-free protein synthesis using a wheat embryo extract.

### BACKGROUND OF THE INVENTION

1. Field of the Invention

The present invention relates to a method for cell-free protein synthesis using a wheat embryo extract under a low temperature condition.

This application claims the priority of Japanese Patent Application No. 2006-056835, which is incorporated herein by reference.

2. Related Background of the Invention

For performing protein synthesis reaction ex vivo, there have been popularly researched various methods, including a so called cell-free protein synthesis method wherein a translation template, amino acids to convert to a substrate, an energy source, various kinds of ions, a buffer and other active factors are added to a cell extract to synthesize a protein in vitro (Patent document Nos. 1-5).

In this cell-free protein synthesis system, the cell extract or the living tissue extract is prepared using a material such as E. coli, wheat embryo or rabbit reticulocyte to synthesize a protein. The cell-free protein synthesis system has excellent advantages comparable to a living cell in two points, “peptide synthesis rate” and “accuracy of translation reaction”. In addition, this system does not require steps of complicated chemical reaction and cumbersome cell culturing. These advantages have accelerated a development to make this system practical. However, usually, the cell extract from an organism is very uncertain in protein synthesis capacity to give a low efficiency of protein synthesis. Further, the cell extract is stored to show a significantly lowered quality. Thus, the cell-free protein synthesis system has provided a product at so small a yield that the product must be labeled with an active isotopic to detect. As a result, this system can not be used as a practical means to produce proteins.

The present inventors have previously provided the following methods to solve drawbacks associated with conventional cell-free protein synthesis system:

(1) a cell extract formulation for cell-free protein synthesis and a method for cell-free protein synthesis (Patent document No. 6), (2) a template molecule having versatility and highly efficient function and a method for cell-free protein synthesis using thereof (Patent document No. 7), (3) diffusive continuous batch method (which may be referred to as a multilayer method) (Patent document No. 8) and (4) repeated batch method (Patent document No. 9)

The synthesis methods described above have exhibited remarkable synthesis efficiencies in comparison to conventional methods for cell-free protein synthesis but there is needed a further improved method for cell-free protein synthesis wherein no additional cost for improved apparatus and a new reagent to add are spent to produce a higher quality protein.

[Non-patent document No. 1] Spirin, A., et al., (1993) Methods in Enzymology, 217, 123-142

[Patent document No. 1] Publication No. JPH6-98790-A

[Patent document No. 2] Publication No. JPH6-225783-A

[Patent document No. 3] Publication No. JPH7-194-A

[Patent document No. 4] Publication No. JPH9-291-A

[Patent document No. 5] Publication No. JPH7-147992-A

[Patent document No. 6] Publication WO00/68412

[Patent document No. 7] Publication WO01/27260

[Patent document No. 8] Publication WO02/24939

[Patent document No. 9] Publication WO2004/097014

### SUMMARY OF THE INVENTION

The subject of the present invention is to provide a method for cell-free protein synthesis with a higher activity more efficiently than the conventional methods for cell-free protein synthesis.

The present inventors examined every condition in conventional methods for cell-free protein synthesis to solve the matters described above.

As a result, the present inventors have found that in a particular method for cell-free protein synthesis, the synthesis is carried out at a temperature of 0° C.-20° C., preferably between 5° C.-20° C., most preferably between 15° C.-20° C., that is, a lower temperature than about 26° C. that is a temperature conventionally known to be suitable, to provide a protein with a high specific activity (a ratio in amount of active protein synthesized to total protein synthesized) at a higher yield, and thus they have completed the present invention.

In summary, the present is as follows;
(1) A method for cell-free protein synthesis, wherein a wheat embryo extract is used under a temperature condition of 0° C.-20° C. to conduct translation reaction, and the method is any one selected from the followings:

1) batch method, 2) dialysis, 3) discontinuous gelfiltration, 4) repeated batch method and 5) repeated-supply batch method.
(2) The method for cell-free protein synthesis according to the preceding aspect 1, wherein the wheat embryo extract is substantially cleared of a contaminating endosperm.
(3) The method for cell-free protein synthesis according to the preceding aspect 1, wherein the wheat embryo extract is substantially cleared of a low-molecular-weight protein synthesis inhibitor.
(4) The method for cell-free protein synthesis according to the preceding aspect 2, wherein the wheat embryo extract is substantially cleared of a low-molecular-weight protein synthesis inhibitor.
(5) The method for cell-free protein synthesis according to the preceding aspect 1, wherein the wheat embryo extract is substantially cleared of an endogenous glycosidase.
(6) The method for cell-free protein synthesis according to the preceding aspect 2, wherein the wheat embryo extract is substantially cleared of an endogenous glycosidase.
(7) The method for cell-free protein synthesis according to the preceding aspect 3, wherein the wheat embryo extract is substantially cleared of an endogenous glycosidase.
(8) The method for cell-free protein synthesis according to the preceding aspect 1, wherein the wheat embryo extract is subjected to any one selected from the followings so that the extract is controlled from sugar phosphorylation system mediated by ATP:

1) removal of monosaccharide, 2) removal of phosphorylated sugar, 3) control of production of monosaccharide from polysaccharide, and 4) control of production of phosphorylated sugar from monosaccharide.
(9) The method for cell-free protein synthesis according to the preceding aspect 2, wherein the wheat embryo extract is subjected to any one selected from the followings so that the extract is controlled from sugar phosphorylation system mediated by ATP:

1) removal of monosaccharide, 2) removal of phosphorylated sugar, 3) control of production of monosaccharide from polysaccharide, and 4) control of production of phosphorylated sugar from monosaccharide.
(10) The method for cell-free protein synthesis according to the preceding aspect 3, wherein the wheat embryo extract is subjected to any one selected from the followings so that the extract is controlled from sugar phosphorylation system mediated by ATP:

1) removal of monosaccharide, 2) removal of phosphorylated sugar, 3) control of production of monosaccharide from polysaccharide, and 4) control of production of phosphorylated sugar from monosaccharide.
(11) The method for cell-free protein synthesis according to the preceding aspect 5, wherein the wheat embryo extract is subjected to any one selected from the followings so that the extract is controlled from sugar phosphorylation system mediated by ATP:

1) removal of monosaccharide, 2) removal of phosphorylated sugar, 3) control of production of monosaccharide from polysaccharide, and 4) control of production of phosphorylated sugar from monosaccharide.

(Effects of the Invention)

The method for cell-free protein synthesis of the present invention can provide protein with a higher specific activity, that is a higher quality by large amount, than the conventional methods for cell-free protein synthesis.

### BRIEF DESCRIPTION OF THE DRAWINGS

FIG. 1 illustrates the results of activity of Luc synthesized under low temperature conditions.

FIG. 2 illustrates the results of specific activity of Luc synthesized under low temperature conditions.

FIG. 3 illustrates the results of amount of Luc synthesized under low temperature conditions.

### DESCRIPTION OF THE PREFERRED EMBODIMENTS

The present invention is a method for cell-free protein synthesis using a wheat embryo extract under a low temperature condition. “Under a low temperature condition” herein means that, at least in a process of a method for cell-free protein synthesis, the wheat embryo extract is set to have a temperature of 0° C.-20° C. in the translation step (so called protein synthesis step), preferably 5° C.-20° C., most preferably 15° C.-20° C. for translation. Meanwhile, the temperature condition in the present invention means a temperature of 0° C.-20° C.

The wheat embryo extract may be set to have the temperature described above in any step before or after a translation template is added. Further, preincubation may be performed under a low temperature condition, but there is no particular limitation. Thus, the wheat embryo extract may be set to be under the temperature condition of the present invention by a well known method per se after the translation template is added. For example, a container holding the wheat embryo extract is left to immerse in a bath set to have a temperature of about 0° C.-about 20° C., allowing the protein synthesis to proceed at a constant temperature for a long period. In addition, the environment itself for protein synthesis is set to have a lowered temperature, allowing the wheat embryo extract to have a temperature set as the temperature condition of the present invention.

Further, the temperature of the wheat embryo extract described above can be determined by a well known measuring method per se. Specifically, a container holding the wheat embryo extract is placed in a temperature controllable bath, which is then adjusted to have almost the same temperature as the extract has, and the temperature can be measured to determine that of the extract. Alternatively, a thermometer is placed directly into the wheat embryo extract to determine.

Proteins (including peptides) synthesized by the method for cell-free protein synthesis of the present invention are not limited in particular. Namely, proteins synthesized under a conventional temperature condition can also be synthesized under the low temperature condition.

The method for cell-free protein synthesis of the present invention is suitable for protein synthesis which are difficult to synthesize under a conventional temperature condition, that is, those derived from organisms adaptable to a low temperature, such as psychrophile (Gerday et al., Trends in Biotechnol. 18 103-107 [2000]).

The methods for cell-free protein synthesis of the present invention using a wheat embryo extract under a low temperature condition include batch method, dialysis, discontinuous gelfiltration, repeated batch method, and repeated-supply method. Dialysis is suitable in particular.

Batch Method

This is a method wherein energy source, amino acids, and tRNA necessary for cell-free protein synthesis are added to a cell extract in a synthesis container (Pratt, J. M. et al., Transcription and Translation, Hames, 179-209, B. D. & Higgins, S. J., eds, IRL Press, Oxford (1984)).

The synthesis container is then adjusted to match a temperature condition of the present invention through a bath to conduct the translation reaction.

Dialysis

Dialysis is a method wherein the permeable membrane of a dialysis apparatus used separates a synthesis reaction solution as the internal liquid from the external liquid to allow mass transfer for protein synthesis (Kigawa et al., The 21st meeting of The Molecular Biology Society of Japan, WID6).

The dialysis container is adjusted to match a temperature condition of the present invention through a bath to conduct the translation reaction.

Discontinuous Gelfiltration

Discontinuous gelfiltration is a method wherein a template RNA, amino acids, and energy sources are supplied to a synthesis reaction system at their respective times as needed while the synthesized products and the degradation products are gel-filtrated at their respective times as needed (Publication No. 2000-333673-A).

The synthesis container is adjusted to match a temperature condition of the present invention through a bath to conduct the translation reaction. The temperature during the gelfiltration is preferably set to the temperature of the present invention.

Repeated Batch Method

Repeated batch method is a synthesis method characterized by making the most effective use of an advantage of the common batch method or the diffusive continuous batch method that a template substance used as a source can be reacted at a high rate in the early stage of synthesis (Patent publication WO2004/097014).

According to the method, before, while or after a reaction gets almost lowered in speed or comes almost stopped, the reaction solution is, repeatedly but discontinuously, either diluted and then concentrated or concentrated and then diluted to allow production of the protein at a large yield.

The synthesis container is adjusted to match a temperature condition of the present invention through a bath to conduct the translation reaction. The reaction solution is preferably concentrated and/or diluted under a temperature condition of the present convention, but there is no particular limitation.

Repeated-Supply Batch Method

According to the method, the reaction solution as reaction phase in a synthesis container is continuously or discontinuously supplied with a feed solution to promote the synthesis. reaction, and then the feed solution is stopped to supply before, while or after the reaction gets almost lowered in speed or comes almost stopped. Subsequently, the reaction solution (reaction phase) is concentrated and then continuously or discontinuously supplied with the feed solution to dilute. The reaction dilution is, repeatedly but discontinuously, concentrated and diluted to allow production of the protein at a large yield (PCT/JP2005/020727).

The synthesis container is adjusted to match a temperature condition of the present invention through a bath to conduct the translation reaction. The reaction solution is preferably concentrated and/or diluted under a temperature condition of the present convention, but there is no particular limitation.

The present invention uses an extract of wheat embryo, which is a embryo of plant seed. As a wheat embryo extract, for example, the attached solution of PROTEIOS™ (from TOYOBO) may be mentioned. Preferably, the wheat seed embryo extract is substantially cleared of a contaminating endosperm component and a low-molecular-weight protein synthesis inhibitor, and further preferably is subjected to removal of endogenous glycosidase, removal of glycolytic enzyme, removal of monosaccharide, removal of phosphorylated sugar, control of production of monosaccharide from polysaccharide, and control of production of phosphorylated sugar from monosaccharide. The extract thus obtained is lowered in amount of components and substances involved in inhibiting the protein synthesis in comparison to conventional wheat seed embryo extracts.

“The wheat seed embryo extract is substantially cleared of a contaminating endosperm” herein means that the extract contains a ribosome which is substantially freed from deadenylation, or is deadenylated at a rate of 7% or less, and preferably 1% or less (see Patent publication WO00/68412).

“The low-molecular-weight protein synthesis inhibitor” herein has a molecular weight of 14,000 Da or less, and can be eliminated from a wheat embryo extract by dialysis using a regenerated cellulose membrane with a molecular weight cutoff of 12,000-14,000 Da. (see Patent publication WO2003/064672).

“The wheat seed embryo extract is substantially cleared of endogenous glycosidase, is substantially cleared of glycolytic enzyme, is substantially cleared of monosaccharide, is substantially cleared of phosphorylated sugar, is subjected to control of production of monosaccharide from polysaccharide, or is subjected to control of production of phosphorylated sugar from monosaccharide” means that sugar and phosphorylated sugar in a cell extract are reduced to a level of 10 mM or less, and preferably to 6 mM or less (in terms of glucose concentration in the extract calculated from an absorbance of 200 OD/ml at 260 nm) (see Patent publication WO2005/063979).

The present invention is explained in detail below with reference to examples, but these examples are not intended to limit the scope of the present invention.

### EXAMPLE 1

Preparation of Wheat Embryo Extract

(1) Preparation of Wheat Embryo

The seeds of Chihoku wheat grown in Hokkaido or Chikugoizumi grown in Ehime were added in a mill (Rotor Speed Millpulverisette type 14 from Fritsch) at a rate of 100 g per minute and gently pulverized at a rotating speed of 8,000 rpm. A fraction containing germinable embryo (mesh size 0.7-1.00 mm) was collected by sieve, and then floated on a mixture of carbon tetrachloride and cyclohexane (a volume ratio of carbon tetrachloride to cyclohexane=2.4:1) to collect a floating fraction containing germinable embryo, which was dried at room temperature to remove the organic solvent and then blown with air at room temperature to remove contaminants such as seed coat, resulting in a crude embryo fraction.

Next, the embryo was screened out from the crude embryo fraction by color difference using a belt type color sorter BLM-300K (supplier: Anzai Manufacturing Co., Ltd., distributor: Anzai Corporation, Ltd.) as follows. This color sorter is an apparatus composed of a means for irradiating a crude embryo fraction with light, a means for detecting reflection light and/or transmission light from the fraction, a means for comparing the measurements and a reference, and a means for selecting one embryo within the reference and removing another embryo without the reference.

The color sorter was supplied with the crude embryo fraction to get 1000 to 5000 grains/cm² on the beige-colored belt, which was then irradiated with light from a fluorescent lamp to detect reflection light. The belt was set to convey at a speed of 50 m/min. A monochrome CCD line sensor (2048 pixels) was used as a light receiving sensor.

First, to eliminate darker components than embryo (such as seed coat), a reference was set between embryo luminance and seed coat luminance, and substances without the reference were removed by aspiration. Then, to screen endosperm out, a reference was set between embryo luminance and endosperm luminance, and substances without the reference were removed by aspiration. The aspiration was conducted using 30 aspirator nozzles installed approximately 1 cm above the conveyor belt (lined at a rate of 1 aspirator nozzle for 1 cm long belt).

This method was repeated to select embryo until a embryo purity of 98% or higher (percent by weight of embryo contained in 1 g of random sample).

The wheat embryo fraction thus obtained was suspended in distilled water at 4° C. and washed using an ultrasonic cleaner until the wash solution became no longer turbid. Then the fraction was suspended in 0.5 vol % solution of Nonidet P40 (from Nacalai Tesque) and washed using the ultrasonic cleaner until the wash solution became no longer turbid, thereby to obtain a wheat embryo. The collected embryo was supplied with twice the volume of an extractant (80 mM HEPES-KOH (pH7.8), 200 mM potassium acetate, 10 mM magnesium acetate, 8 mM dithiothreitol, 4 mM calcium chloride, each 0.6 mM of 20 kinds of L-amino acids and 2.5 mM ATP) relative to the embryo by wet weight, and was limitedly homogenized three times using Waring blender at 5,000-20,000 rpm for every 30 seconds.

(2) Preparation of S-30 Fraction Using Precipitation Auxiliary Agent

20 wt % of sea sand or swollen particles of Sephadex G25 were added to the obtained homogenate (homogenized product) described above and mixed. The sea sand was previously treated as follows before added to the homogenate: washed with water→washed with 5-hold volume of 0.1 N NaOH or KOH→washed with water→washed with 0.1 N HCl→washed with water→heated at 100-120° C. for RNase inactivate and then subjected to drying.

The homogenate mixed with the sea sand was centrifuged twice at 30,000×g for 30 minutes, subsequently once for 12 minutes, to get a translucent supernatant (S-30 fraction). The obtained S-30 fraction was applied on Sephadex G25 which had been equilibrated with an eluent (40 mM HEPES-KOH (pH7.8), 200 mM potassium acetate, 10 mM magnesium acetate and 4 mM DTT), gel-filtrated to eliminate a low molecular substance having a molecular weights of 1000 Da or less, thereby to prepare a embryo extract.

### EXAMPLE 2

Constructing Translation Template for Luciferase (Luc)

There was constructed pEU vector in which a base sequence encoding Luc (pSP-luc+: from Promega, Cat. No. E1781) and E01 sequence were introduced. Subsequently, the pEU vector was used as a template to prepare its transcription template by PCR. The transcription template was added to a transcription reaction solution [80 mM HEPES-KOH (pH7.8), 16 mM magnesium acetate, 10 mM dithiothreitol, 2 mM spermidine, 2.5 mM 4NTPs (4 kinds of nucleotide triphosphates), 0.8 u/μl RNase inhibitor, 1.6 u/μl SP6 RNA polymerase in their respective final concentrations] and transcripted at 37° C. for 3 hours, thereby to construct a translation template (the translation template was constructed according to the method described in Proc. Natl. Acad. Sci. USA, 2002, vol 99, p 14652-14657: Sawasaki, T et al.).

### EXAMPLE 3

Synthesis of Luciferase (Luc) by Dialysis Under Different Temperature Conditions

62.6 μl of a reaction solution for protein synthesis containing 25 μl of the embryo extract prepared in Example 1 was prepared, wherein the reaction solution was composed of 29 mM HEPES-KOH (pH7.8), 95 mM potassium acetate, 2.7 mM magnesium acetate, 0.4 mM spermidine (from Nacalai Tesque), 20 kinds of L-amino acids respectively at 0.23 mM, 2.9 mM dithiothreitol, 1.2 mM ATP (from Wako Pure Chemical Industries, Ltd.), 0.25 mM GTP (from Wako Pure Chemical Industries, Ltd.), 15 mM phosphocreatine (from Wako Pure Chemical Industries, Ltd.) and 0.46 μg/l creatine kinase (from Roche) in their respective final concentrations. This reaction solution was supplied with the translation template for Luc constructed in Example 2 at 16 μg/50 μl and then poured into a container provided with a dialysis membrane. The container was submerged to allow dialysis in a solution which had the same composition as the above described reaction solution for protein synthesis except for no embryo extract contained, and incubation was conducted for 24 hours under different temperature conditions as follows.

The Luc was synthesized under each temperature condition of 0, 5, 10, 15, 20 and 26° C., and detected in activity. Further, the synthesized Luc and a standard Luc were subjected to native polyacrylamide gel electrophoresis respectively, and stained with CBB to get their respective bands, their staining intensities of which were then compared to calculate the amount of the synthesized Luc. These results were totalized to calculate the specific activity of Luc.

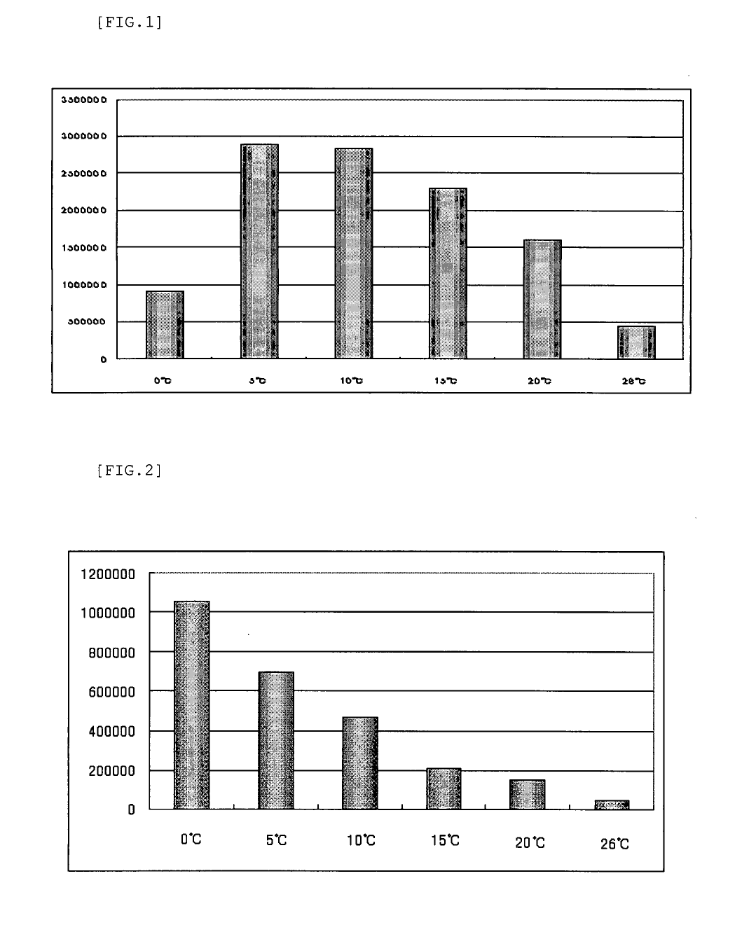

FIG. 1 illustrates that any temperature condition of 0, 5, 10, 15 and 20° C. of the present invention gave approximately 2 to 7 times higher Luc activity than the temperature condition of 26° C.

Further, FIG. 2 illustrates that any temperature condition of 0, 5, 10, 15 and 20° C. of the present invention gave approximately 3 to 30 times higher specific activity of protein than the temperature condition of 26° C. This demonstrates that the present invention can synthesize a highly active protein at a remarkably high rate compared with conventional methods.

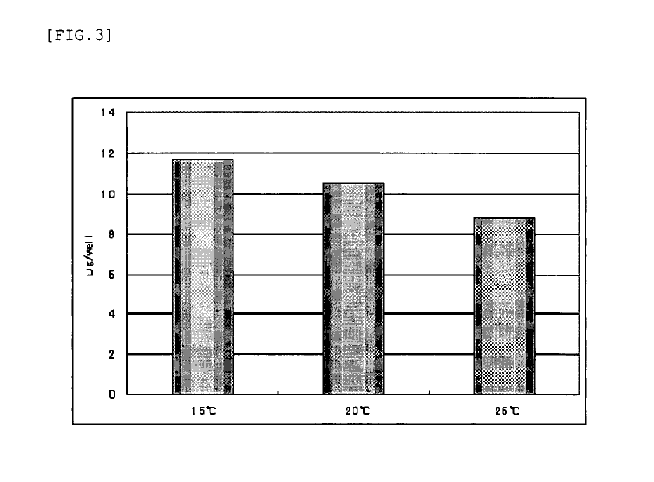

Further, FIG. 3 demonstrates that protein synthesis at 15° C. produced a significantly increased amount of protein compared with conventional synthesis at 26° C., indicating that the present invention contributes to an increased amount of protein synthesized.

From the facts above, it may be concluded that the method for cell-free protein synthesis of the present invention can synthesize a protein with a higher specific activity by a larger production amount than a conventional method conducted under a common temperature condition.

### INDUSTRIAL APPLICABILITY

The present invention provides a method for cell-free protein synthesis which needs no new apparatus and reagent to allow synthesis of a protein with a high specific activity.

What is claimed is:

1. A method for cell-free protein synthesis, wherein a wheat embryo extract is used under a temperature condition of 0° C.-20° C. to conduct translation reaction, and the method is any one selected from the followings:
1) batch method, 2) dialysis, 3) discontinuous gelfiltration, 4) repeated batch method and 5) repeated-supply batch method.

2. The method for cell-free protein synthesis according to claim 1, wherein the wheat embryo extract is substantially cleared of a contaminating endosperm.

3. The method for cell-free protein synthesis according to claim 1, wherein the wheat embryo extract is substantially cleared of a low-molecular-weight protein synthesis inhibitor.

4. The method for cell-free protein synthesis according to claim 2, wherein the wheat embryo extract is substantially cleared of a low-molecular-weight protein synthesis inhibitor.

5. The method for cell-free protein synthesis according to claim 1, wherein the wheat embryo extract is substantially cleared of an endogenous glycosidase.

6. The method for cell-free protein synthesis according to claim 2, wherein the wheat embryo extract is substantially cleared of an endogenous glycosidase.

7. The method for cell-free protein synthesis according to claim 3, wherein the wheat embryo extract is substantially cleared of an endogenous glycosidase.

8. The method for cell-free protein synthesis according to claim 1, wherein the wheat embryo extract is subjected to any one selected from the followings so that the extract is controlled from sugar phosphorylation system mediated by ATP:
1) removal of monosaccharide, 2) removal of phosphorylated sugar, 3) control of production of monosaccharide from polysaccharide, and 4) control of production of phosphorylated sugar from monosaccharide.

9. The method for cell-free protein synthesis according to claim 2, wherein the wheat embryo extract is subjected to any one selected from the followings so that the extract is controlled from sugar phosphorylation system mediated by ATP:
1) removal of monosaccharide, 2) removal of phosphorylated sugar, 3) control of production of monosaccharide from polysaccharide, and 4) control of production of phosphorylated sugar from monosaccharide.

10. The method for cell-free protein synthesis according to claim 3, wherein the wheat embryo extract is subjected to any one selected from the followings so that the extract is controlled from sugar phosphorylation system mediated by ATP:
1) removal of monosaccharide, 2) removal of phosphorylated sugar, 3) control of production of monosaccharide from polysaccharide, and 4) control of production of phosphorylated sugar from monosaccharide.

11. The method for cell-free protein synthesis according to claim 5, wherein the wheat embryo extract is subjected to any one selected from the followings so that the extract is controlled from sugar phosphorylation system mediated by ATP:
1) removal of monosaccharide, 2) removal of phosphorylated sugar, 3) control of production of monosaccharide from polysaccharide, and 4) control of production of phosphorylated sugar from monosaccharide.
